SEQUENCE LISTING (2 sequences)

SEQ ID NO: 1 - DNA, 22 bp, Unknown
  Ficticious example provided on page 10 of specification.
tagagagagagagatcatcgat

SEQ ID NO: 2 - DNA, 32 bp, Unknown
  Ficticious example provided on page 11 of specification.
gagtgtatctgcccgccggcgtgcccggctac

(19) United States
(12) Patent Application Publication
(10) Pub. No.: US 2005/0227278 A1
(43) Pub. Date: Oct. 13, 2005

(54) Recursive categorical sequence assembly

(75) Inventor: Michael Wall, Vacaville, CA (US)

Correspondence Address: LARGE SCALE BIOLOGY CORPORATION, 3333 VACA VALLEY PARKWAY, SUITE 1000, VACAVILLE, CA, 95688, US

(73) Assignee: LARGE SCALE BIOLOGY CORPORATION, Vacaville, CA (US)

(21) Appl. No.: 11/139,463

(22) Filed: May 26, 2005

Related U.S. Application Data
(63) Continuation of application No. 10/043,377, filed on Jan. 11, 2002.

(51) Int. Cl. C12Q 1/68; G06F 19/00; G01N 33/48; G01N 33/50; C12P 19/34

(52) U.S. Cl. 435/6; 435/91.2; 702/20

(57) ABSTRACT

The present invention provides a method for efficiently creating assemblies. The present invention also provides a web-based system for scientists to interact with a computer to implement the method. Further the scientist is able to upload and download information to and from the method to and from a database. The present invention also provides an efficient hardware architecture to implement the method.

### FIELD OF INVENTION

The field of the present invention is in the area of high-speed and high-throughput computing in the area of biotechnology. Specifically the invention is related to computing assemblies for sets of DNA or RNA sequence reads.

### BACKGROUND

1. DNA/RNA Sequence Reads

Reads are the scientific results of DNA or RNA materials that are run on gels or some other means to determine the genetic material's nucleotide sequence. Each read possesses at least two different types of data. The first part is the base call of the read. The base call may be a best guess of the base (adenine, guanine, cytosine, or tyrosine) in a particular position in the genetic material being sequenced. The second part may be a generated probability that the particular base call is correct (sometimes referred to as a Phred scored).

2. Assemblies

Assemblies are reads that have been put together in a manner similar to a jigsaw puzzle. Each read may be a relatively small part of a larger portion of genetic material. The reads may be overlapping and these overlapping portions of the reads may be used to splice the reads together. Further, mismatches are allowed when overlapping these reads due to the probabilities assigned to each base in each read. If a base call contains a low probability read it may be likely that it will be removed or changed when spliced together with other reads. In this way assemblies are created by putting reads together. Phrap is the current industry standard method of creating assemblies from reads. Other assembly programs include SEQMAN II (from DNA Star) and GCG (from Accelrys) as well as assemblers created by TIGR and STADEN.

3. Phrap

Phrap is a computer program developed at the University of Washington to create assemblies from sets of DNA or RNA sequence reads. Any other program designed to create assemblies from reads may also be incorporated with the present invention. Phrap was originally created as a tool for the scientists of the Human Genome Project who needed to join reads together into assemblies. The algorithm as taught be the creators of Phrap used in the current method of Phrap is as follows (http://www.phrap.org/phrap.docs/phrap.html):
a) The procedure used in both Phrap and cross_match to find sequence matches is the following. First, (in Phrap only) any region at the beginning or end of a read that consists almost entirely of a single letter is converted to ‘N’s; such regions are highly likely to be of poor data quality which if not masked can lead to spurious matches. Reads are then converted to uppercase (in order to allow case-insensitive word matches). All matching words of length at least minmatch between any pair of sequences are then found, by (i) constructing a list of pointers to each position (in each sequence) that begins a word of at least minmatch letters not containing ‘N’ or ‘X’; (ii) sorting the list (using a modified version of quicksort, with string comparison as the comparison function+a few tricks to improve speed by keeping track of the parts of the words that are known already to match); (iii) scanning the sorted list to find pairs of matching words. For each such pair, a band of a specified width (in the imaginary dot matrix for the two sequences), centered on the diagonal defined by the matching words, is defined. Overlapping bands (for the same pair of sequences) are merged. Following construction and merging of bands, a “recursive” SWAT search of each band is used to find matching segments with score greater than or equal to minscore: “recursive” here means that if such a match is found, the corresponding aligned segments in each sequence are (conceptually) X'd out and the process repeated on the remaining portions (if any) of each sequence. This procedure allows (at the cost of some redundant calculation) detection of multiple matching pieces in different locations, and will usually find most copies of repeats (since they generally occur in separate bands). By default, SWAT scores are complexity-adjusted (so that matches for which the collection of matching nucleotides has biased composition have their scores significantly penalized.)
b) In Phrap, sequence pairs with score >=minscore are considered for possible merging. A critical issue here is the appropriate score matrix for SWAT. [Given the generally high accuracy of reads and the desirability of minimizing false matches due to imperfect repeats, a relatively high penalty seems desirable. Currently I use +1 for a match, −9 for a mismatch involving A,C,G, or T,0 for a match or mismatch involving N, −1 for a match or mismatch involving X, −11 for the gap initiating penalty (the first residue in a gap), and −10 for the gap extension penalty (each subsequent residue).] Setting the indel penalties slightly higher than the mismatch penalties gives better alignments by favoring mismatches in compression regions. Since SWAT uses profiles, one has the option of distinguishing different quality levels by use of different symbols (e.g. upper case for more accurate calls, lower case for less accurate calls) and setting the penalties appropriately; this would involve using the quality levels to adjust the symbols used in the reads, which should be done AFTER the word matching routine. (At present it does not seem particularly useful to use differing positive scores for different nucleotides to reflect their different frequencies). 1a) Determine “confirmed” part of each read (i.e. the part which appears in a SWAT alignment against some other read; a read with the same name—up to an internal ‘.’ if any—is not considered confirming). Probable chimeras are detected as reads for which the confirmed part can be separated into two non-overlapping pieces, separated by at most MAX_CHIMERA_GAP bp (currently 30) such that the part confirmed by a given read lies in one or the other piece but not both; AND such that each piece has a prematurely terminating alignment with some other read. Chimeric reads may arise from i) chimeric clones; ii) gel mistracking across lanes; iii) unremoved sequencing vector; (iv) deletion clones. Reads having two non-overlapping confirmed pieces (but failing the “premature termination” condition) are often non-chimeras that are the only link between two non-overlapping contigs, and thus should be permitted in the assembly. Identify “strongly confirmed” regions in each read: having matches to a reverse sense read. Identify deletions. Identify “rejected” alignments—those which don't extend as far as they should, or that have mismatches involving high quality bases.
c) Sort all matching pairs by decreasing LLR score, and assemble layout by progressively merging pairs with high score. Consistency of merge is required: the SWAT alignment implies relative offset of one contig with respect to another, and hence a potential implied overlap. The SWAT alignment(s) should extend over essentially the entire region of implied overlap, apart from an allowable gap (necessary to allow for the fact that the ends of the alignments are somewhat uncertain). Probable deletion clones (identified as reads which have two adjacent pieces matching two separated pieces of another read, and having no confirming read across the breakpoint), and chimeras are not used in any merges. [N.B. Following is obsolete: Potential chimeras are deferred to a second pass (as well as being flagged in output), so that true reads will assemble first.]
d) Construct contig “consensus” as a mosaic of individual reads. Strategy: ends of alignments, and midpoints of perfectly matching segments of sufficient length, define crosslinks—pursue crosslinks which increase accuracy and extend read. Formally this is done by constructing a weighted directed graph whose nodes consist of (selected) positions in reads; there are bidirectional edges with weight 0 between aligned bases in overlapping reads, and unidirectional edges from 5′ to 3′ positions within a single read, with weight equal to the total quality of the sequence between the two nodes. Standard C.S. algorithms (dating back to Tarjan) permit identification of a path with maximal weight, in time linear w.r.t. number of nodes. The quality values for the resulting sequence are inherited from the read segments of which it is composed
   4. Current uses of Phrap and Other Assemblers on Computers

Phrap and other assembly software are currently employable on Unix and PC based computers. Currently Phrap's limitations are based on the computer's ability to perform the algorithm. Specifically, as the number of reads goes up by an order of N, the numbers of computations needed by Phrap goes up by an order of N². Currently to run Phrap with high numbers of reads companies and scientists have tried to use bigger computers to solve the problem. However, there has been little work done to alter the Phrap algorithm itself.

5. Divide and Conquer Computer Methods—as Described by Cormen, Leiserson, and Rivest in “Introduction to Algorithms”.

Many useful algorithms are recursive in structure: to solve a given problem, they call themselves recursively one or more times to deal with closely related subproblems. These algorithms typically follow a divide-and-conquer approach: they break the problem into several subproblems that are similar to the original problem but smaller in size, solve the subproblems recursively, and then combine these solutions to create a solution to the original problem.

The divide and conquer paradigm involves three steps at each level of the recursion:
a. Dividing the problem into a number of subproblems.
b. Conquering the subproblems by solving them recursively. If the subproblem sizes are small enough, however, just solve the problem in a straightforward manner.
c. Combining the solutions of the subproblems into the solution for the original problem.
   6. Myers and Miller Sequence Alignment Algorithm

Myers and Miller is one of many sequence alignment algorithms. The algorithm contains many attractive computational characteristics. In particular, the Myers and Miller algorithm is dramatically more memory efficient than traditional methods such as the Needleman-Wunsch and Smith-Waterman.

To align two sequences of length N and M, both the Needleman and Wunsch and Smith Waterman require a memory resource proportional to N×M while Myers and Miller requires a memory section proportional to N. This becomes very important when N and M grow large.

Using graph theory, Myers and Miller were also able to improve the time scaling characteristics, a full exposition of which is beyond the scope of this text. See Myers and Miller CABIOS (1989) and “Bioinformatics and Genome Analysis” by David. W. Mount (in preparation). The Myers and Miller algorithms is well know and popular and is found in numerous applications such as: EMBOSS Stretcher, Fasta Align, ClustalW and GNU diff.

7. Compression Ratio

For a sequence A, the compression ratio is defined as the length of the compressed sequence of A divided by the length of the original sequence A. The compression ratio may be computed after using any string/sequence compression method such as the Lempel-Ziv algorithm or the Burros-Wheeler algorithm to compress the sequence A. String compression is well known in the art of computer science.

### OBJECTS AND SUMMARY OF PRESENT INVENTION

It may be an object of the present invention to create and implement a method for creating an assembly. The method may possess several steps. These steps may include obtaining a set of DNA or RNA sequence reads, grouping the DNA or RNA sequence reads into categories, or running an assembly program on each of the separate categories of DNA or RNA sequence reads. The grouping and running steps may also be executed recursively as necessary.

It may also be an object of the present invention to construct an apparatus for creating assemblies from DNA or RNA sequence reads. The apparatus may have several functions performed by various elements. For instance, there may be means for obtaining a set of DNA or RNA sequence reads, means for grouping DNA or RNA sequence reads into categories, and means for creating assemblies from the DNA or RNA sequence reads.

A further object of the present invention may be a computer system employing a graphical user interface or command line interface including a display device and a selection device, a method of displaying information on the display device in a menu form, accepting menu selection input from a user, and a method of processing data. The method of processing data may include retrieving a set of menu entries for the menu, each of the menu entries representing a method to perform upon DNA or RNA sequence reads, displaying the set of menu entries on the display device or displaying a set of parameters on the display device. The method may also include providing the user an opportunity to modify the set of parameters, receiving an indication of a menu entry selection from the user via the selection device, or in response to the indication of a menu entry selection, performing a method on the DNA or RNA sequence reads to create assemblies based on the set of parameters and said set of menu entries.

Another object of the present invention may be a set of application program interfaces embodied on a computer-readable medium for execution on a computer in conjunction with an application program that determines assemblies of DNA or RNA sequence reads. The application program interface may include a first interface that receives function selections for a method for assembling DNA or RNA sequence reads, a second interface that may receive parameters for the functions, a third interface that may receives DNA or RNA sequence reads, and an interface that may return an assembly of the DNA or RNA sequence reads.

It may also be an object of the present invention to create a divide and conquer algorithm to improve Phrap and other assembly methods. This method may divide DNA or RNA sequence reads into various groups by a variety of functions. These groups may then have assemblies created by Phrap or another assembler. These assemblies may then be used to create assemblies for all of the reads. The method may perform more than one level of recursion.

### BRIEF DESCRIPTION OF DRAWINGS

FIG. 1 is an exemplary bin selection method based on number of bases in a read.

FIG. 2 is an exemplary bin selection method based on percentile location of a read.

FIG. 3 is an exemplary flow chart of the method used to create assemblies by the present invention.

FIG. 4 is an exemplary web-page interface that may be used in conjunction with the present invention.

FIG. 5 is an exemplary data storage unit capable of storing the present invention

### DETAILED DESCRIPTION OF VARIOUS EMBODIMENTS

The present invention can be embodied as a software application resident with, in, or on any of the following: a database, a Web-server, a separate programmable device that communicates with a Web-sever through a communication means, a software device, a tangible computer-usable medium, or otherwise. Embodiments comprising software applications resident on a programmable device are preferred. Alternatively, the present invention can be embodied as hardware with specific circuits, although these circuits are not now preferred because of their cost, lack of flexibility, and expense of modification.

The present invention may be a computer program used in conjunction with Phrap or any other sequence assembly method. The computer program may be written in Perl, C, or any other language. A computer program may be joined with Phrap or any other sequence assembly program or run on top of it. A computer program may also be written to replace Phrap and determine sequence assemblies on its own. The present invention may be used to separate the reads into categories. These groups may then individually be used as input to Phrap, to another program, or to a separate module of the present invention. The resulting assemblies of Phrap, the other program, or separate module of the present invention are then combined into new sets of “reads” to be possibly used again as input to a recursion of Phrap, the other program, or separate module of the present invention.

The present invention may provide several ways to break the original sets of reads into separate categories. These sets may be created heuristically or by specific functions. The following are several functions that may be uses to create category groupings among the reads.

1. By Size

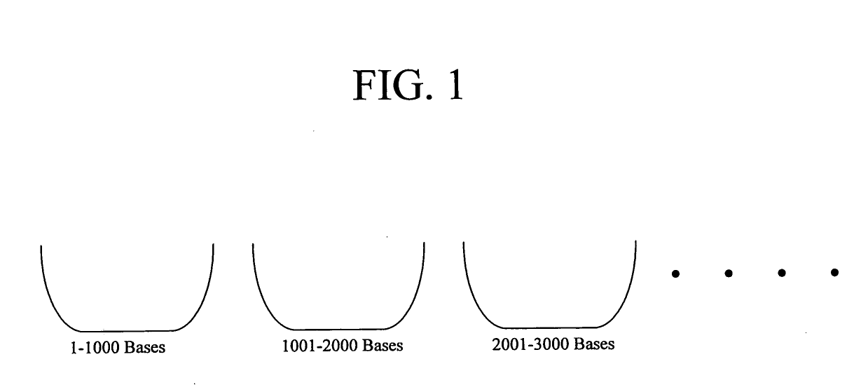

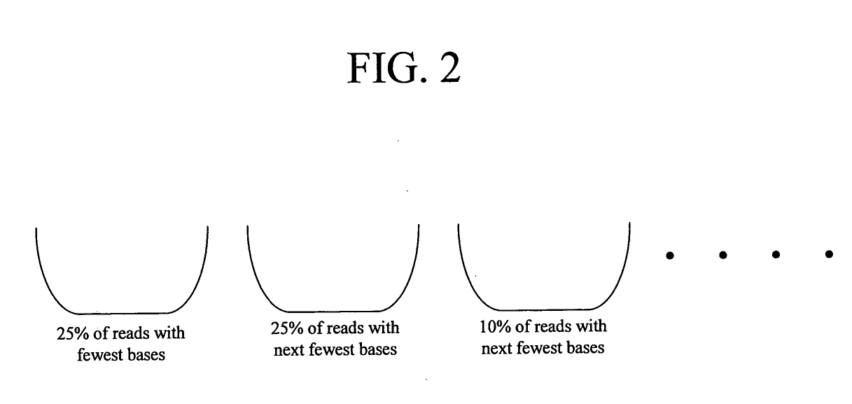

The reads may be broken up by size in a variety of ways. First, the size groupings may be on relative size. For instance the reads can be broken up into equal grouping where bins all contain the same number of reads (FIG. 1). Second the bins can be of varying size and be based on the sizes of reads (FIG. 2). For instance all reads of 400-500 base pairs may go into a single bin. Other methods of creating bins based on the size of the read may include other concepts such as deviation from mean or any other measurement based upon the size or relative size of the read.

2. By Entropy

Entropy is defined as −(Σp_k*log(p_k)). p_k, the probability of a given base occurring is defined as: (number of occurrences of base k)/(sequence length). Other methods of creating bins based on the entropy of the read may include other concepts such as deviation from mean or any other measurement based upon the entropy or relative entropy of the read.

3. By GC Percentage

GC percentage is defined as (Σguanine+Σcytosine)/(Σtotal bases). The algorithm may cluster the reads by GC percentage. The size of the bins may be based on the number of reads in each bin or the GC percentage. Other methods of creating bins based on the GC percentage of the read may include other concepts such as deviation from mean or any other measurement based upon the GC percentage or relative GC percentage of the read.

4. By Nature of Longest Repeat

In each read there may be some longest repeat. The repeat may be of a single base or a repetition of multiple bases. The algorithm may cluster each of the reads based on the longest repeat of a single base or the longest repeat of multiple bases. A repeating region is well know in the art. As an example, the sequence “TAGAGAGAGAGAGATCATCGAT” contains a GA repeat from bases three through sixteen, which is this sequence's longest repeat. The categories of the present invention may be based on the GC percentage or any other percentage of the repeat as well as similar methods.

5. By Nature of Longest High or Low Entropy Area

Each read may have a long high or low entropy area within the read's sequence. This area will have some GC content. An example of a sequence with high entropy and high GC content is “GAGTGTATCTGCCCGCCGGCGTGCCCGGCTAC”. The entropy is high because there is not an even distribution of A, G, C, and T. The GC percentage is high because over 70% percent of the sequence is a G or a C.

Using this method to divide reads, the reads may be placed into bins based on the GC content of the area of the longest high or low entropy read. Also an average of some number of the highest or lowest entropy areas may be used to categorize the data.

6. Information Carrying Capacity (Compression Ratio)

Each read may carry some amount of information. In a random ordering each strand may exhibit base probabilities close to ¼ for each base. Compression algorithms exploit information within a sequence to compress the sequence. The more information in the sequence, the more the sequence may be able to be compressed. Therefore sequences with more information will tend to be compressed further, as their compression ratio may exhibit. Further if sequences carry similar amounts of information then they may contain a similar structure.

The compression ratio may then be used to determine possible sequence similarity. The compression ratio may provide information about the information carrying content of reads. Reads with similar compression ratios may then be assumed to have a higher probability of possessing overlapping regions. Further, the information content may be able to take into account reads of different lengths by providing a metric that may accurately describe the complexity of each read. Therefore, compression ratios may be used to divide the reads into subgroups.

7. Compression of Appended Sequences

A different scheme to create the sequence grouping may be with a method that uses the compression ration of appended reads to create groupings. For instance two reads A and B may be tested to see if they should be placed into the same grouping. The test is conducted by compressing two sequences and comparing their results. The two sequences A and B are appended to form the sequence AB. The sequence AB is then compressed. If the AB compression ratio is high relative to the compression ratios of sequences A and B then sequences A and B may be placed into the same grouping. If the AB compression ratio is low relative to the compression ratios of A and B then the sequences A and B may not be placed into the same category.

A second scheme to create sequence grouping with the compression of appended sequences may be with a method that determines if compressions are high or low by using the metric of the compression ratio of AB subtracted by the compression ratio of AA. If this metric is below some threshold value then the sequences A and B may be placed into the same grouping. If the metric is above some threshold value then the sequences A and B may be placed into a separate grouping. In this way sequences can be compared to one another and placed into grouping. Whenever, a sequence is found to not fit into any of the current groupings, it is placed into a new grouping.

A second method may also be used to create new groupings for the first or second schemes to create sequence groupings through the compression of appended sequences. A single sequence is used to create the first grouping. After the best sequences based on the compression ratios, numbering some pre-determined (by-user or automatic) number, are placed into a single group based on the selected first sequence, a new sequence is selected from the group of sequences that are not yet in a group. Then the best sequences relative to this newly selected sequence are placed into a group. This process may continue until no sequence is not within a group.

8. Hybrid

Two or more of any of the above functions may be combined to create groupings. In this way an even better refinement of groupings may be possible.

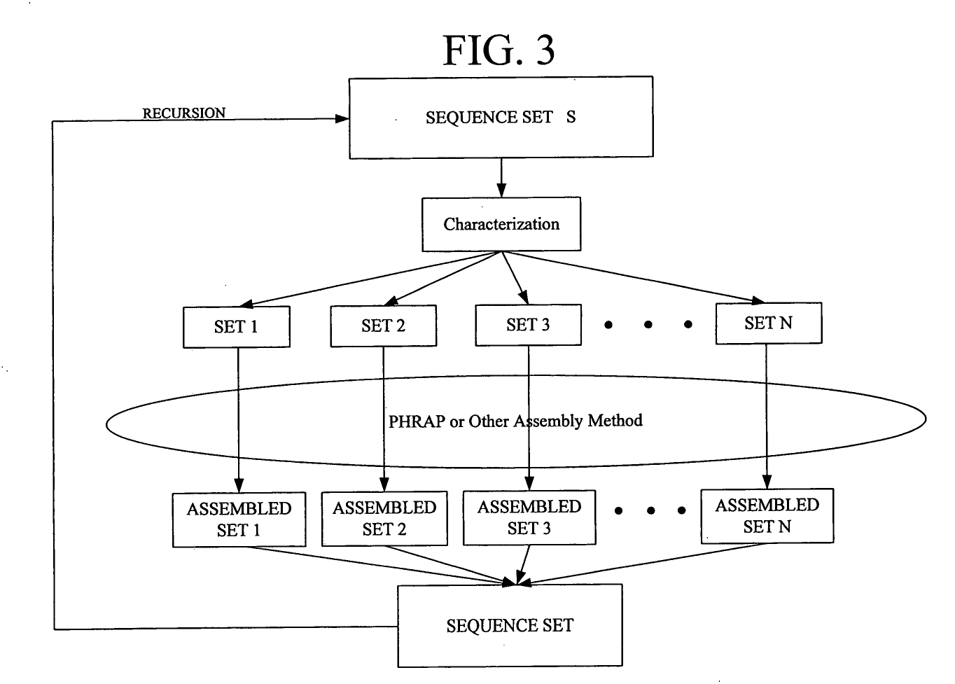

The present invention may be executed in a fashion described in FIG. 3. The present invention will begin with the input of a set of sequences or reads 301. The present invention may then categorize the reads. By selecting one of the above methods to categorize the read to determine the bins (such as 302, 303) in which to place each read. After the reads are placed into separate bins, Phrap or other assembly method is executed on each bin. The result is a set of assemblies (such as 304 and 305). These assemblies may be further placed into used as input for the sequence set 301 as the recursive step. Then the reads will be placed into categories. Once the final assembly is created then the algorithm is finished and no further recursions are executed.

The algorithm used for Phrap contains approximately N² computations, where N is the number or reads to be assembled. However, the algorithm of the present invention contains approximately C*I*M² computations. In the present invention, C is defined as the number of categories assembled individually, I is the number of iterations or recursions in the algorithm, and M is approximately the average number of sequence per category. Since M will tend to be much less than N (unless most of the reads are placed in the same category) the algorithm of the present invention may tend to run faster and need less computations as the number of reads increases.

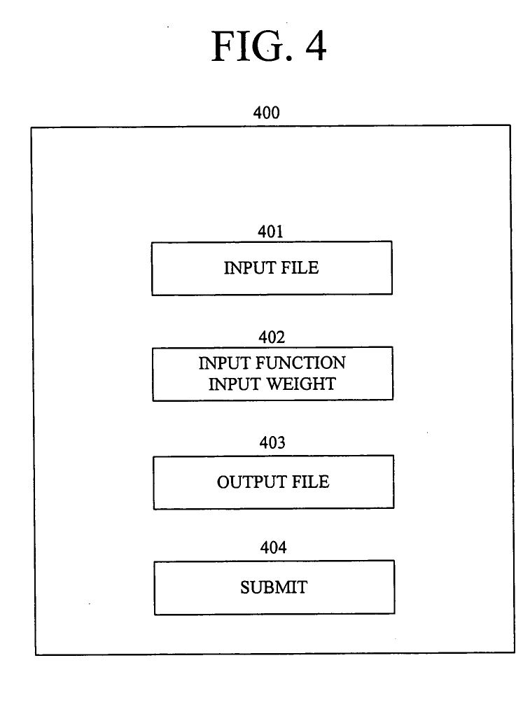

The algorithm may be executed through a web-page and web-server. An exemplary display of such a web-page is FIG. 4. The web-page of FIG. 4 allows a user to input the parameters of the an assembly creator consistent with the present invention. Bar 401 is an exemplary input to the display of a file or reads. It consists of an input bar where a user may type in a file containing reads to be assembled. The input bar 401 may possess the ability to specify more than one read file. Bar 402 is an exemplary input bar of the function to be used to determine the division of the reads into grouping of reads. The input bar may be able to specify more than one function to be used. Separate input bars or input bar 402 may also be used to weigh the amount each function is used to determine which subgroup a read is categorized into. Input bar 403 allows for input of the destination file for the assembly created by the algorithm. Submit button 404 may cause the computer to execute the algorithm with the given parameter. After completing the algorithm the computer may save the results to the file specified in input bar 403. It may also crate a new web page or display that graphically or textually displays the resulting assembly to the user. Such a graphical display of an assembly might be with the use of Consed. An alternative embodiment may be the use of the present algorithm with a command line interface instead of a GUI interface.

The preferred embodiment is for the present invention to be executed by a computer as software stored in a storage medium. The present invention may be executed as an application resident on the hard disk of a PC computer with an Intel Pentium or other microprocessor and displayed with a monitor. The processing device may also be a multiprocessor. The computer may be connected to a mouse or any other equivalent manipulation device. The computer may also be connected to a view screen or any other equivalent display device.

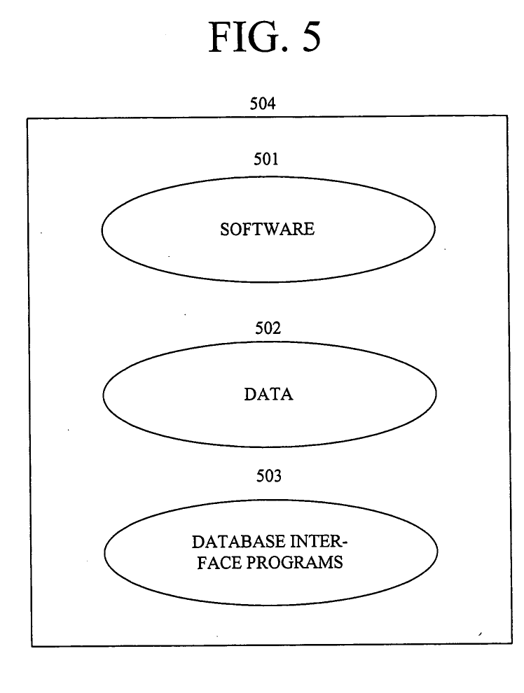

Referring to FIG. 5, part of the process analyzing reads to create assemblies may be executed by the assembly creation code (software) 501 stored on the program storage device 504. This code may access the read data 502 and database interface programs 503. Further a GUI within a program or associated with a web-based application may be used to interact with the assembly program.

FIG. 5 shows a program storage device 504 having storage areas 501-503. Information is stored in the storage area in a well-known manner that is readable by a machine, and that tangibly embodies a program of instructions executable by the machine for performing the method of the present invention described herein for creating assemblies from read data. Program storage device 504 could be volatile memory, such as dynamic random access memory or non-volatile memory, such as a magnetically recordable medium device, such as a hard drive or magnetic diskette, or an optically recordable medium device, such as an optical disk. Alternately, other types of storage devices could be used.

In the current embodiment, a user may execute a plurality of functions, some of which are shown in FIG. 3, to process read data. The functions allow the user to create assemblies from the read data. In other embodiments, other functions may be employed.

The embodiments described herein are merely illustrative of the principles of this invention. Other arrangements and advantages may be devised by one skilled in the art without departing from the spirit or scope of the invention. Accordingly, the invention should be deemed not to be limited to the above detailed description. Various other embodiments and modifications to the embodiments disclosed herein may be made by those skilled in the art without departing from the scope of the following claims.

1. A method for creating an assembly comprising:
(a) obtaining a set of DNA or RNA sequence reads;
(b) grouping the DNA or RNA sequence reads into categories;
(c) running an assembly program on each of the separate categories of DNA or RNA sequence reads; and
(d) repeating steps (b) and (c) as necessary.

2. The method of claim 1 wherein the grouping of the DNA or RNA sequence reads is done based on a function.

3. The method of claim 2 where said function is based on the size of the reads.

4. The method of claim 2 where said function is based on the entropy of the reads.

5. The method of claim 2 where said function is based on the GC percentage of the reads.

6. The method of claim 2 where said function is based on the nature of the longest repeat of the reads.

7. The method of claim 2 where said function is based on the nature of the one or more regions of highest entropy of the reads.

8. The method of claim 2 where said function is based on the nature of the one or more regions of lowest entropy of the reads.

9. The method of claim 2 where said function is based on the compression ratio of the reads.

10. The method of claim 2 where said function is based on the compression ratio of appended sequences of the reads.

11. The method of claim 2 where said function is a hybrid of two or more functions based on the qualities of the reads.

12. An apparatus for creating assemblies from DNA or RNA sequence reads comprising:
(a) means for obtaining a set of DNA or RNA sequence reads;
(b) means for grouping DNA or RNA sequence reads into categories; and
(c) means for creating assemblies from the DNA or RNA sequence reads.

13. In a computer system having a graphical interface including a display device and a selection device, a method of displaying information on the display device in a menu form and accepting menu selection input from a user, the method comprising:
retrieving a set of menu entries for the menu, each of the menu entries representing a method to perform upon DNA or RNA sequence reads;
displaying the set of menu entries on the display device;
displaying a set of parameters on the display device;
providing the user an opportunity to modify said set of parameters;
receiving an indication of a menu entry selection from the user via the selection device; and
in response to said indication of a menu entry selection, performing a method on'the DNA or RNA sequence reads to create assemblies based on said set of parameters and said set of menu entries.

14. A set of application program interfaces embodied on a computer-readable medium for execution on a computer in conjunction with an application program that determines assemblies of DNA or RNA sequence reads, comprising:
a first interface that receives functions for a method for assembling DNA or RNA sequence reads;
a second interface that receives parameters for said functions;
a third interface that receives DNA or RNA sequence reads; and
returns an assembly of said DNA or RNA sequence reads.

15. A method for assembling sequence reads, comprising the steps of:
a) categorizing a plurality of sequence reads into at least two sub-groups of sequence reads based on an identifiable characteristic of the sequence reads in each sub-group;
b) matching sequences reads within each sub-group thereby creating assemblies of said sequence reads within each respective sub-group; and
c) repeating steps a) and b) with all unassembled sequence reads and newly created assemblies.

16. A method as set forth in claim 15, wherein said categorizing step includes identifying sequence reads having similar sizes.

17. A method as set forth in claim 15, wherein said categorizing step includes identifying sequence reads having similar entropies.

18. A method as set forth in claim 15, wherein said categorizing step includes identifying sequence reads having similar GC percentages.

19. A method as set forth in claim 15, wherein said categorizing step includes identifying sequence reads having similar longest repeats.

20. A method as set forth in claim 15, wherein said categorizing step includes identifying sequence reads having similar natures of regions of high entropy.

21. A method as set forth in claim 15, wherein said categorizing step includes identifying sequence reads having similar natures of regions of low entropy.

22. A method as set forth in claim 15, wherein said categorizing step includes identifying sequence reads having similar compression ratios.

23. A method as set forth in claim 15, wherein said categorizing step includes identifying sequence reads having compression ratios after sequence appending.

24. A method as set forth in claim 15, wherein said categorizing step includes identifying sequence reads having two or more similar characteristics.

25. A method as set forth in claim 15, wherein said categorizing step includes identifying sequence reads having
